(19) United States
(12) Patent Application Publication
(10) Pub. No.: US 2015/0366586 A1
(43) Pub. Date: Dec. 24, 2015

(54) Foot Rake

(71) Applicant: Richard James White, Winfield (CA)

(72) Inventor: Richard James White, Winfield (CA)

(21) Appl. No.: 14/307,769

(22) Filed: Jun. 18, 2014

(51) Int. Cl. A61B 17/54

(52) U.S. Cl. CPC A61B 17/54; A61B 2017/320008

(57) ABSTRACT

The ‘Foot Rake’ is a hand held foot care and cleaning device specifically designed to efficiently, effectively and safely remove thick skin buildup from the soles of feet, after first softening the feet by soaking in a temperate or warm saponaceous or Epsom Salt solution.

### BACKGROUND

Since the middle of the twentieth century, the trend for personal hygiene has moved away from bathing to showering, but unfortunately with that change, our feet have lost the benefit of a good, skin softening soak. Periodic soaking is essential for the health and condition of the feet, and to remove hard skin from the soles. Soaking the feet prior to cleaning, aids tremendously in the removal of hard skin buildup.

Without that essential soak, many people find their neglected feet accumulate a thickening crust of unsightly, uncomfortable, hard dry dead skin, and if left will eventually crack and cause painful splits into the soft tissue of the feet.

Without pre-softening the feet by soaking, hard skin buildup is very difficult to remove. Feet do get wet in a shower, and do become cleaner and fresher, but showering does not soak the feet, nor does it aid removal of thick skin buildup. Soap helps soften skin, but again without a soak, does not soften sufficiently to remove skin buildup, and furthermore as a slipping hazard, it is very inadvisable to soap feet while in a shower.

Responding to the need to remove hard skin, multitudes of foot care products are sold in the marketplace, and in review, every single one of these products is either inefficient, ineffective, or carries a risk of injury or consequence to the user. There is not one single foot care device or product on the market today, recognized as the preferred method or treatment for removing hard skin buildup from feet.

Without efficient, effective and safe foot care remedies, people fail to give their feet the attention they need, and in severe cases of neglect, their feet can deteriorate to terrible conditions with thick dry flaking skin, which may crack and cause painful splits into soft skin tissue. Many so desperate for foot care, may resort to extreme solutions like shaving their feet with a razor, or sanding them with a power tool such as a belt sander.

Another problem confronting many wishing to apply a personal foot care treatment to their feet, is their limitation of reach. The soles of our feet are not the easiest place to access, and with advancing age, it becomes more and more difficult for many to do anything to their feet.

Normal healthy skin is moist, and under normal conditions does not split. When feet are neglected the skin thickens and forms a dry crust, which will eventually crack, a natural process where the body is attempting to shed unwanted dead skin. Cracks and splits develop mostly at pressure points such as the heel and ball of the foot, and when splits into soft tissue become painful, that is when many are finally prompted to apply some kind of care to their suffering feet. Unfortunately when thick cracked skin is removed from the feet, the exposed sub-layer of skin being thinner and weaker, may split further and bleed before the injured foot eventually heals.

The essence of the Foot Rake invention is to provide a better means to remove hard skin, by connecting the benefit of soaking the feet, and using the Foot Rake to remove hard skin buildup more efficiently, effectively and safely than any other known foot care device, method or treatment. To substantiate these claims, we will review all known foot care devices and methods, marketed or devised to remove hard skin buildup from feet.

### PRIOR ART

Before soaps were invented or were available, the Romans had the ‘Strigil,’ a hand held metal hook device used primarily after bathing to strip dirt from the pre-oiled skin of the torso and limbs. In its application, the ‘Strigil,’ is the closest known personal hygiene device or instrument to the concept of the Foot Rake.

Patent searches found a hand held foot care device called a ‘Foot Care Instrument,’ filed under Canadian Patent 1127933. This device consists of a handle with a roller used to position a sharp cutting blade to the skin. Compared to the Foot Rake, the ‘Foot Care Instrument’ not intended or considered for use after a softening pre-soak of the feet, and radically compensates for dealing with hard skin by utilizing a sharp cutting blade, making this ‘Instrument’ dangerous and risky to use.

### Current Foot Care Devices and Methods

Typically, foot treatments are either wet or dry, and to rid dead skin from the feet, the current known methods and devices involve cutting, shredding, abrading, sanding, brushing, scraping, fish nibbling and chemicals. The hand held Foot Rake utilizing a skin softening pre-soak together with a stroking or raking motion, is a unique device and method, and the best way to remove unwanted skin from the feet.

Foot care devices made with sharp metal cutting blades, are an obvious hazard. Applying a sharp blade to the bottom of the feet is awkward and lacks control, so nicks and cuts should be expected, and when such injuries occur, they are indeed painful. Prone to aggravation at the pressure points, nicks and cuts may open and bleed, and of course risk infection. There is also a secondary risk from using a foot care device with sharp metal cutting blades, when the device is accidently dropped and possibly causing injury to the user, or damage to bathroom fixtures.

Shredding foot care devices with several shallow angle cutting edges intended to shred off skin like a kitchen grater, can gouge and groove the skin leaving it tender and rough to snag clothing and hosiery, and furthermore, grooved skin has thinner, weaker spots prone to drying out and splitting.

Foot care products that utilize abrasion as their method, are usually made from either rock based materials or metal. When used wet, fine grade abrasive foot care products are hindered by loss of friction. Pumice or other silica rock products can smooth skin to a limited degree, but cannot remove significant thick skin buildup, and when used dry, these products quickly glaze over with removed skin cells. When coarse grade filing or abrasive devices are used wet, they are also hindered by loss of friction, and when used dry they produce messy dust, or may inflict friction burns, or become clogged and ineffective, and if hard skin is forcibly stripped dry from the feet, the newly exposed hydrated skin not only resists further removal, but within a day or so, rapidly dries to form more dry skin.

Many wet application foot care devices take the form of elaborate floor standing devices, most intended for use in the shower. Typically these devices are supposed to clean the feet with various driven or non-driven brushes, or stationary scraping or abrasive surfaces. Such foot care devices are really overboard in their concept and are not found by the consumer to be desirable. In their crude approach, these devices often expose soft skin tissue to excessive abrasion. They are bulky, inconvenient, awkward to store, use or accommodate in the bathroom, and are a tripping hazard, and because of their impracticality and cost, such devices have no position in the marketplace.

‘Garra rufa’ or ‘Doctor fish,’ are used to nibble hard skin buildup from feet immersed in a tank containing these animals, but considered as a possible source of contamination from one person to another, the use of these fish for cleaning feet is prohibited in many jurisdictions, and they are certainly not practical for home use.

Chemical preparations have been developed to remove hard skin from feet, and work something like paint stripper. The feet are first soaked in the chemical preparation for a short period, then over the next week or so, the dead skin loosens and has to be repeatedly picked off the feet. Although effective in the end result, this foot care treatment is expensive, slow, time consuming, inconvenient and very messy with dead skin shedding into footwear, bedding, and elsewhere.

The hand held Foot Rake as designed, gives the user excellent control to rake and remove only pre-softened dead skin, but if inadvertently applied to normal healthy skin, there is no consequence, because the Foot Rake cannot cut, injure, damage or gouge soft live healthy tissue when stroked along the feet, and the skin will be left clean and smooth, and the feet invigorated and comfortable.

### Description of the Foot Rake Invention

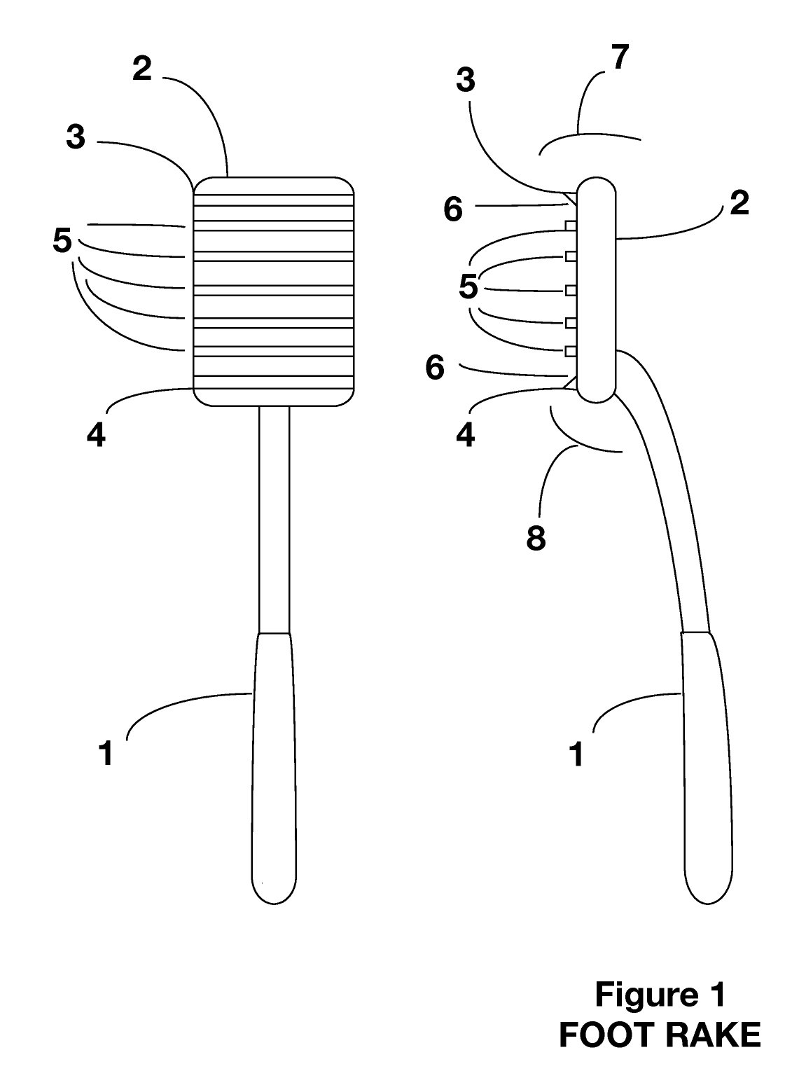

Referring to FIG. 1, the Foot Rake is a personal handheld foot care device used to stroke, rake and remove hard skin from the feet, after first preparing the hard skin for removal by soaking in a warm saponaceous or Epsom Salt solution. Handle (1) is connected to the rake head (2), which by means of its design, contains, retains or holds, and enables one or more rake strips illustrated in three basic design options and shown as details (3, 4 & 5), to be presented and stroked along the feet, to rake away unwanted hard skin buildup.

The Foot Rake as described in this claim, is intended for use only after the feet are first prepared by soaking, and under this condition, the Foot Rake as described, will provide the best, most efficient, most effective, and safest foot care for removal of hard skin from the soles of the feet.

With its fundamental handle design with a nominal length of 12 inches, the Foot Rake empowers the user to reach the soles of their feet and apply the required stroking and raking motion of the rake head, and indeed a Foot Rake handle of more than 12 inches in length, similarly empowers users with reduced reaching ability.

There are three basic stroking motions to using the Foot Rake, and they are:
A) Stroking the Foot Rake backwards with the handle raised at an angle to the skin, so only the outer end rake strip (3) contacts and rakes the skin. This stroking motion is good for raking the soles of the feet.
B) Stroking the Foot Rake forwards with the handle level with the skin, so only the inner end rake strip (4) contacts and rakes the skin. This stroking motion is good for raking the heels of the feet.
C) Stroking the Foot Rake backwards and forwards, so the center rake strips (5) contact and rake the skin. This stroking motion is good for raking all parts of the feet with thicker skin buildup, and to give a smoothing finish to the skin.

The Foot Rake performance, capability and stroking method will vary with the configuration, number of rake strips, rake strip profile, and the material or materials from which the rake strips are made. The Foot Rake user will have a choice of rake strip, and will be able to select the rake strip design found most suitable for their personal needs, such as skin smoothing or more aggressive thick skin removal. Rake strips when made as separate component parts, will be shaped and profiled to facilitate their attachment to the rake head, and be designed and made from materials to best suit the users foot care needs.

All rake strips whether made as separate or integral parts of the rake head, are also shaped and profiled with rake faces set at a suitable rake angle (6), for efficient, effective and safe removal of unwanted skin material. The center rake strips (5) in FIG. 1, are shown with a 90° rake angle, but this angle can be varied to best suit the raking function, the rake strip materials, and the spacing between rake strips, but in general, the rake angle provides for the best angle to provide the most effective raking of dead skin.

To facilitate use of the outer rake strip (3), clearance space (7) provides for when the Foot Rake handle is inclined from the skin surface. To facilitate use of the inner rake strip (4), used particularly at corner locations such as the heels of the feet, clearance space (8) provides for clearance of the handle with the feet.

### Foot Rake Options

The basic Foot Rake consists of three component parts comprising the handle, the rake head, and rake strip. The handle is attached to the rake head, and the rake head contains one or more rake strips which are stroked across the soles of the feet to rake and remove dead skin cells softened either from soaking while bathing, or after soaking in a warm saponaceous or Epsom Salt solution.

The Foot Rake may also consist of more than the three basic component parts with the addition of various permanently attached or removable brushes, pads, sponges or other application devices or materials, or whatever is deemed suitable as a vehicle for applying creams or other foot treatment preparations for either softening the skin of the feet in preparation for raking, or moisturizing after raking, or applying any other foot care treatment or preparation.

The three basic component parts of the Foot Rake can be made as one piece with the handle integral with the rake head, and the rake head integral with one or more rake strips.

The three basic component parts of the Foot Rake can also be made in more than one piece with the handle integral with the rake head, and separately made rake strips permanently attached to the rake head either by adhesion, riveting, welding, snap fit, or some other physical or chemical means.

The three basic component parts of the Foot Rake can also be made in two pieces with the handle removable from a rake head with integral rake strips.

The handle of the Foot Rake can be made with two rake heads, both either permanently attached or detachable from each or either end of the handle.

A rake head of the Foot Rake can be made to contain just one rake strip, or made to contain two or more rake strips with equal or varying spaces between them.

The rake head of a Foot Rake made with integral rake strips, can used as a disposable unit for hygienic purposes, such as when used by health spars, podiatrists, chiropodists, or other communal applications, to avoid contamination from one person to another.

The three component parts of the Foot Rake can also be made in more than two pieces with the handle integral with the rake head, and the rake head made with detachable and therefore replaceable rake strips.

The three component parts of the Foot Rake can be made in separate pieces with the handle and the rake strips detachable from the rake head, so all component parts can be replaceable and made from different materials.

The handle length of the Foot Rake is typically 8 to 12 inches (20 to 30 cm), to facilitate users with normal reaching ability to rake the soles of their feet.

The handle length of the Foot Rake can be more than 12 inches (30 cm), to facilitate users with limited reaching ability to rake the soles of their feet, and the handle can also be made extendable to facilitate all users.

The handle of the Foot Rake will have sufficient strength and rigidity to apply adequate raking pressure to remove soak softened skin cells from the feet.

The width of the Foot Rake rake head will typically from less than 1 inch, up to more than 3 inches (2 to 9 cm), to accommodate sufficient lengths of rake strip to efficiently and effectively rake the feet.

The raking faces of the Foot Rake rake strips can have many and various profiles, and be shaped to best suit the materials from which they are made, the desired raking duty, and other properties or personal raking preferences.

The rake strips for the Foot Rake can be made and mounted so the raking faces of the rake strips point towards the handle for use when pulling, and away from the handle when pushing. The rake strips can also be made with dual raking faces, enabling the Foot Rake to be used in both the pulling and pushing directions of the handle.

The rake strips for the Foot Rake can be made with various raking profiles, including but not limited to concave, beaded, ridged, and burred rake faces, using such manufacturing processes as molding, forming, extruding or machining.

The rake strips for the Foot Rake when intended to be removable and replaceable from the rake head, can be made with various cross-sections to suit retaining grooves slotted across the rake head, enabling retention and replacement of the rake strips.

The handle for the Foot Rake can be made from plastic, wood, bamboo, metal, or any composition, composite or combination of these materials.

The rake head for the Foot Rake can be made from plastic, rubber, wood, bamboo, or metal, or any composition, composite or combination of these materials.

The rake strips for the Foot Rake can be made from plastic, rubber, wood, bamboo, ceramic, pumice, emery, silica, polymer, or cast, rolled, drawn or formed metal, or any composition, composite or combination of these materials.

The Foot Rake when not made from hard and heavy materials such as metal or silica based products, has the benefit of reduced personal injury or damage risk from dropping, corrosion, cutting and scratching.

The Foot Rake when used with non metallic rake strips, is a considerable safety improvement over foot care devices which use sharpened metal blades that can cut healthy skin, or injure by penetrating cracked dead skin or split healthy skin tissue.

The Foot Rake used on wet skin softened by pre-soaking, is an improvement over abrasion devices used dry to rub, file or sand feet, and which may impart dry friction burns.

The Foot Rake when used on wet skin softened by pre-soaking, is an improvement over dry abrasion devices used to rub, file or sand feet, because newly exposed hydrated skin resists further removal by dry abrasion.

The Foot Rake when used on wet skin softened by pre-soaking, is an improvement over dry abrasion devices used to rub, file or sand feet, because newly exposed hydrated skin rapidly dries to produce more unwanted dry skin.

The Foot Rake using rake strips on wet skin softened by pre-soaking, is an improvement over dry abrasion foot care devices made from pumice, emery, silica or other stone materials, which clog, glaze and cease to continue removing unwanted skin.

The Foot Rake using rake strips on wet skin softened by pre-soaking, is an improvement over wet abrasion foot care devices made from pumice, emery, silica or other stone materials, which lose friction and cease to continue removing unwanted skin.

The Foot Rake using rake strips on wet skin softened by pre-soaking, is an improvement over foot care devices intended to shred, shave or grate skin, but which may gouge and groove, leaving the skin rough to snag clothing and hosiery, and be more prone to drying and splitting.

The open design of the Foot Rake provides for more efficient cleaning after use compared to any other foot care device, because the material it removes from the feet is soft and soluble, and easily rinses away.

### Foot Rake Marketing Options

The Foot Rake can be sold as a stand alone foot care device, or sold with various preparations for pre-soaking the feet prior to raking, and with various preparations for treating the feet after raking, or any combination of these options.

The Foot Rake can be sold as an integral one-piece item, a multi piece item, or as individual separate components, with various combinations of replaceability, size, design, and material options for the handle, rake head and rake strips.

1. A Foot Rake foot care device wherein a handle is embodied to a rake head having one or more laterally placed permanent or replaceable non-abrasive Rake Strips, for stroking the soles of wet feet, to rake and remove dead skin cells softened from bathing or soaking in a warm saponaceous or Epsom salt solution.

2. The Foot Rake of claim 1, wherein the Rake Strip is a single or double sided cross-section, configured straight, angled, bowed or contoured across the width of a rake head, typically but not limited to 1 inch to 3 inches (2 cm to 8 cm) wide, for presenting of the Rake Strip to the skin at a minimum rake angle of 90°.

3. The Foot Rake of claim 2, wherein the Rake Strip is manufactured from plastic, rubber, wood, bamboo, metal or any non-abrasive composite or combination of these materials.
